(19) United States
(12) Patent Application Publication
(10) Pub. No.: US 2021/0228660 A1
(43) Pub. Date: Jul. 29, 2021

(54) USE OF PROTEASOME INHIBITOR AND ALPHAVIRUS IN PREPARATION OF ANTI-TUMOR MEDICAMENT

(71) Applicant: GUANGZHOU VIROTECH PHARMACEUTICAL CO., LTD., Guangzhou, Guangdong (CN)

(72) Inventors: Guangmei YAN, Guangzhou, Guangdong (CN); Haipeng ZHANG, Guangzhou, Guangdong (CN); Wenbo ZHU, Guangzhou, Guangdong (CN); Yuan LIN, Guangzhou, Guangdong (CN); Shoufang GONG, Guangzhou, Guangdong (CN); Jiankai LIANG, Guangzhou, Guangdong (CN); Jing CAI, Guangzhou, Guangdong (CN); Chunqiang LIU, Guangzhou, Guangdong (CN)

(21) Appl. No.: 17/057,489

(22) PCT Filed: May 22, 2019
(86) PCT No.: PCT/CN2019/087977

§ 371 Date: Nov. 20, 2020

(30) Foreign Application Priority Data
May 22, 2018 (CN) 201810493340.0

(51) Int. Cl. A61K 35/768; A61K 45/06

(52) U.S. Cl. CPC A61K 35/768; A61K 45/06

(57) ABSTRACT

Use of a proteasome inhibitor and an alphavirus in the preparation of an anti-tumor medicament. The proteasome inhibitor can be used to prepare an alphavirus anti-tumor synergist. A pharmaceutical composition comprising a proteasome inhibitor and an alphavirus, including a pharmaceutical kit comprising the proteasome inhibitor and the alphavirus, and use of the proteasome inhibitor and the virus in the treatment of tumors, particularly tumors insensitive to the alphavirus.

### TECHNICAL FIELD

The invention belongs to the field of biomedicine, and relates to use of a combination of a proteasome inhibitor and an alphavirus in preparation of anti-tumor medicament.

### BACKGROUND

Oncolytic virus is a kind of replicable virus that selectively infects and kills tumor cells without damaging normal cells. Oncolytic virus therapy is an innovative tumor targeted therapy strategy, which uses natural or genetically engineered viruses to selectively infect tumor cells and replicate in tumor cells to achieve targeted lysis and killing of tumor cells without damaging normal cells.

M1 virus (Alphavirus M1) belongs to the genus Alphavirus, and has good application effect in the preparation of anti-tumor medicament. For example, Chinese invention patent application 201410425510.3 discloses that M1 virus can selectively cause tumor cell death without affecting normal cell survival, which has a very good application prospect in anti-tumor field. However, different tumors have different sensitivity to M1 virus. For example, as disclosed in the Chinese invention patent application 201410425510.3, when M1 is used as an anti-tumor medicament, the effect on colorectal cancer, liver cancer, bladder cancer and breast cancer is less obvious than that on pancreatic cancer, nasopharyngeal cancer, prostate cancer and melanoma; glioma, cervical cancer and lung cancer are the second; while gastric cancer is the least significant.

Screening compounds that increase the therapeutic effect of oncolytic virus tumor is expected to increase the anti-tumor spectrum and anti-tumor intensity of oncolytic virus. In the patent CN201510990705.7 previously applied by the inventor, chrysophanol and derivatives thereof are used as anti-tumor synergist for oncolytic viruses, and the combination of the two can reduce the viability of tumor cells to 39.6%. At present, the mechanism of the combined application is not clear, nor is it known which other substances, such as oncolytic viruses, have been reported to have synergistic effects with them, and to what extent.

It is not easy to study the synergistic pathway of specific oncolytic viruses. Although there are many substances that have been reported to have anti-tumor synergistic effects on some oncolytic viruses, it is difficult to predict the ways of synergism because different oncolytic viruses often have different synergistic mechanisms.

Medicaments have different effects on the replication of different viruses or the anti-tumor immune response of the body, and its mechanism is very complicated. At present, numerous substances with synergistic effect on specific oncolytic virus have been studied and developed, but they often only have positive effects on some viruses, have no effect on other viruses, or even bring negative effects, which brings a great challenge to the development of oncolytic virus synergist.

For alphaviruses, the development of synergistic pathways is also facing the same problem. For example, as a HDAC inhibitor that has been shown to have a synergistic effect on oncolytic rhabdovirus (Nguye, T. L., et al., Chemical targeting of the innate antiviral response by histone deacetylase inhibitors renders refractory cancers sensitive to viral oncolysis. Proceedings of the national academy of sciences, 2008. 105(39): p. 14981-14986.; Shulak, L., et al., Histone Deacetylase Inhibitors Potentiate Vesicular Stomatitis Virus Oncolysis in Prostate Cancer Cells by Modulating NF-kB-Dependent Autophagy. Journal of Virology, 2014. 88(5): p. 2927-2940.; Bridle, B. W., et al., HDAC Inhibition Suppresses Primary Immune Responses, Enhances Secondary Immune Responses, and Abrogates Autoimmunity During Tumor Immunotherapy. Molecular therapy, 2013. 21(4): p. 887-894.), the inventors found that it did not achieve a similar synergistic effect when used in combination with alphavirus. This is also one of the reasons why it is difficult to develop alphavirus synergist.

### SUMMARY

The invention aims to provide an alphavirus anti-tumor synergist.

Another object of the present invention is to provide an anti-cancer synergist capable of selectively enhancing the killing effect of alphavirus on tumor cells without affecting normal cells.

Another object of the present invention is to provide the use of a proteasome inhibitor in the preparation of an alphavirus anti-tumor synergist.

It is another object of the present invention to provide an anti-tumor pharmaceutical composition which enables alphavirus to exert a better anti-tumor effect.

Another object of the present invention is to provide an alphavirus synergistic medicament which is safe and effective against tumors insensitive to alphavirus.

Another object of the present invention is to provide a more accurate and safer synergistic therapy for oncolytic viruses.

The invention finds that the proteasome inhibitor can enhance the anti-tumor effect of alphaviruses.

The invention provides a combination composition of a proteasome inhibitor and an alphavirus and use thereof in preparation of anti-tumor medicament.

Proteasome is a multi-catalytic complex ubiquitous in eukaryotes. Proteasome is the main tool for cells to regulate specific proteins and remove misfolded proteins, which is responsible for the rapid degradation of unwanted or damaged target proteins. Proteasome inhibitors can block the degradation of a large number of regulatory proteins, cause intracellular signal system disorder and overload, lead to cell growth inhibition, and eventually delay or even stop the tumor progression. A number of proteasome inhibitors, including Bortezomib, have been used in clinical treatment of malignant tumors, and the effect is significant.

The sedimentation coefficient of proteasome density gradient centrifugation is 26S, so it is also called 26S proteasome. 26S proteasome consists of one 20S core particle and one or two 19S regulatory particles. The 20S core particle is a 720 kDa hollow barrel subcomplex composed of two outer a rings and two inner 13 rings. Each layer ring is composed of seven closely related subunits and can be expressed as α₁₋₇β₁₋₇β₁₋₇α₁₋₇. The active site of proteasome is located in the lumen of 20S core particle, and a unique residue catalytic site is formed by β subunit Thr1. Three of the seven β subunits have catalytic activity due to the presence of Thr1: β₁ (Gene ID: 5689), β₂ (Gene ID: 5690) and β₅ (Gene ID: 5693).

Preferably, the proteasome inhibitor is an inhibitor of the core particle of the proteasome.

As a more preferred embodiment, the proteasome inhibitor is an inhibitor inhibiting subunit β₁, β₂ or β₅.

The proteasome inhibitor refers to a substance that inhibits proteasome activity, or inhibits the activity or expression of any one of the subunits, or blocks assembly of the subunits, or degrades the proteasome.

The proteasome inhibitors include the proteasome inhibitors disclosed up to now, as well as proteasome inhibitors that have been developed in the future to have similar functions.

The inventors have experimentally verified that the oncolytic effect of alphavirus can be significantly enhanced by inhibiting the proteasome. The inventors have used a proteasome inhibitor (e.g., Bortezomib) in combination with an alphavirus (e.g., M1 virus) to act on tumor cells and found that the proteasome inhibitor can enhance anti-tumor effects in combination with the alphavirus.

Proteasome inhibitors, such as Bortezomib, have been studied as one of the medicaments whose effects on different oncolytic viruses vary. It has been reported that Bortezomib can enhance the oncolytic effect of some viruses, such as vesicular stomatitis virus (VSV) (Yarde D N, Nace R A, Russell S J. Oncolytic vesicular stomatitis virus and bortezomib are antagonistic against myeloma cells in vitro but have additive anti-myeloma activity in vivo. Exp Hematol. 2013 December; 41(12):1038-49.), HSV-1 (Suryadevara C M, Riccione K A, Sampson J H. Immunotherapy Gone Viral: Bortezomib and oHSV Enhance Antitumor NK-Cell Activity. Clin Cancer Res. 2016 Nov. 1; 22(21):5164-5166.; Yoo J Y, Jaime-Ramirez A C, Bolyard C, Dai H, Nallanagulagari T, Wojton J, Hurwitz B S, Relation T, Lee T J, Lotze M T, Yu J G, Zhang J, Croce C M, Yu J, Caligiuri M A, Old M, Kaur B. Bortezomib Treatment Sensitizes Oncolytic HSV-1-Treated Tumors to NK Cell Immunotherapy. Clin Cancer Res. 2016 Nov. 1; 22(21):5265-5276. AND Yoo J Y, Hurwitz B S, Bolyard C, Yu J G, Zhang J, Selvendiran K, Rath K S, He S, Bailey Z, Eaves D, Cripe T P, Parris D S, Caligiuri M A, Yu J, Old M, Kaur B. Bortezomib-induced unfolded protein response increases oncolytic HSV-1 replication resulting in synergistic antitumor effects. Clin Cancer Res. 2014 Jul. 15; 20(14):3787-98.), gamma herpesvirus (GHVs) (Jiang H, Clise-Dwyer K, Ruisaard K E, Fan X, Tian W, Gumin J, Lamfers M L, Kleijn A, Lang F F, Yung W K, Vence L M, Gomez-Manzano C, Fueyo J. Delta-24-RGD oncolytic adenovirus elicits anti-glioma immunity in an immunocompetent mouse model. PLoS One. 2014 May 14; 9(5): e97407.). The mechanism of this synergistic oncolytic effect may be related to proteasome inhibitors to increase oncolytic virus replication or enhance the body's anti-tumor immune response.

At the same time, proteasome inhibitors have been reported to have inhibitory effects on the replication of a variety of viruses, suggesting that proteasome inhibitors may have side effects in the therapeutic application of oncolytic viruses and cannot synergize these oncolytic viruses. For example, the proteasome inhibitor MG132 can reduce avian reovirus replication and virus-induced apoptosis (Chen Y T, Lin C H, Ji W T, Li S K, Liu H J. Proteasome inhibition reduces avian reovirusreplication and apoptosis induction in cultured cells. J Virol Methods. July 2008; 151 (1): 95-100.); Proteasome inhibitors significantly inhibit vesicular stomatitis virus (VSV) protein synthesis, virus accumulation and protect infected cells from the toxic effects of VSV replication, delaying the replication of poliovirus (NeznanovN, Dragunsky E M, Chumakov K M, Neznanova L, Wek R C, Gudkov A V, Banerjee A K. Different effect of proteasome inhibition on vesicular stomatitis virus and poliovirus replication. PLoS One. 2008 Apr. 2; 3(4): e1887.); Proteasome inhibitors also inhibit HIV-1 virus replication (Yu L, Mohanram V, Simonson O E, Smith C I, Spetz A L, Mohamed A J. Proteasome inhibitors block HIV-1 replication by affecting both cellular and viral targets. Biochem Biophys Res Commun. 2009 Ju117; 385(1):100-5.).

The present invention found for the first time that the proteasome inhibitor can be used as an anti-tumor synergist/drug resistance reversing agent of the alphavirus.

The invention provides a use of proteasome inhibitor in preparation of alphavirus anti-tumor synergist/drug resistance reversing agent.

The drug resistance reversing agent means that when some alphaviruses are used as anti-tumor medicament for treating tumors, some tumors are less sensitive to alphaviruses, or the tumors are resistant to alphaviruses, and in this case, an alphavirus combination with a proteasome inhibitor (as a drug resistance reversing agent) can be used to reverse the resistance of the tumors to the alphaviruses.

The proteasome protein inhibitor includes, but is not limited to, a compound selected from the group consisting of or a derivative thereof having proteasome inhibitory effect, or a pharmaceutically acceptable salt, solvate, tautomer, isomer thereof: Bortezomib, Carfilzomib, MG-132, ONX-0914, ONX-0912 (Oprozomib), CEP-18770 (Delanzomib), MLN-9708 (Ixazomib), Epoxomicin, VR23, MLN-2238, Celastrol, PI-1840. The compounds may be obtained by, but are not limited to: chemically isolated or synthesized by itself or purchased commercially.

In a preferred example of the invention, the proteasome protein inhibitor is Bortezomib, Carfilzomib CEP-18770, MLN-9708, ONX-0912, or a combination thereof.

In a preferred example of the invention, the proteasome protein inhibitor is Bortezomib, which has the structural formula shown in Formula 1:

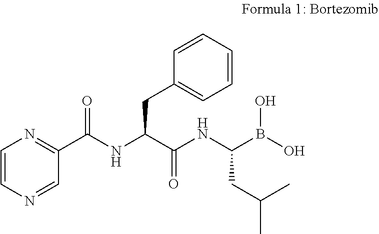 [SMILES: CC(C)C[C@H](NC(=O)[C@H](Cc1ccccc1)NC(=O)c1cnccn1)B(O)O]

In another preferred example of the invention, the proteasome protein inhibitor is Carfilzomib, which has the structural formula shown in Formula 2:

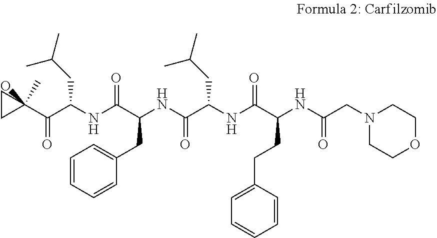

In another preferred example of the invention, the proteasome protein inhibitor is Oprozomib (ONX-0912), which has the structural formula shown in Formula 3:

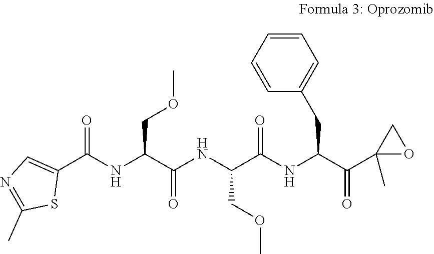 [SMILES: COC[C@H](NC(=O)c1cnc(C)s1)C(=O)N[C@@H](COC)C(=O)N[C@@H](Cc1ccccc1)C(=O)C1(C)CO1]

In another preferred example of the invention, the proteasome protein inhibitor is Delanzomib (CEP-18770), which has the structural formula shown in Formula 4:

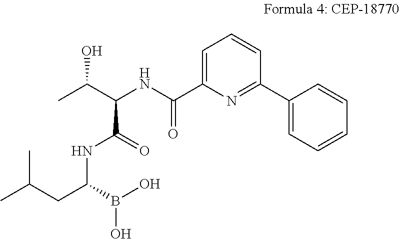 [SMILES: CC(C)C[C@H](NC(=O)[C@H](NC(=O)c1cccc(-c2ccccc2)n1)[C@H](C)O)B(O)O]

In another preferred example of the invention, the proteasome protein inhibitor is MLN-9708, which has the structural formula shown in Formula 5:

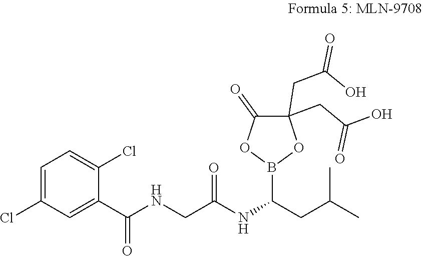 [SMILES: CC(C)C[C@H](NC(=O)CNC(=O)c1cc(Cl)ccc1Cl)B1OC(=O)C(CC(=O)O)(CC(=O)O)O1]

In another preferred example of the invention, the proteasome protein inhibitor is MG-132, which has the structural formula shown in Formula 6:

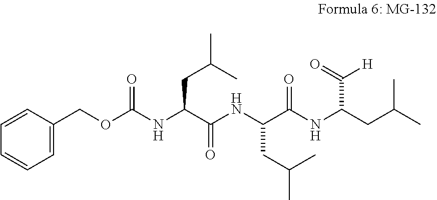 [SMILES: CC(C)C[C@@H](C=O)NC(=O)[C@H](CC(C)C)NC(=O)[C@H](CC(C)C)NC(=O)OCc1ccccc1]

In another preferred example of the invention, the proteasome protein inhibitor is ONX-0914, which has the structural formula shown in Formula 7:

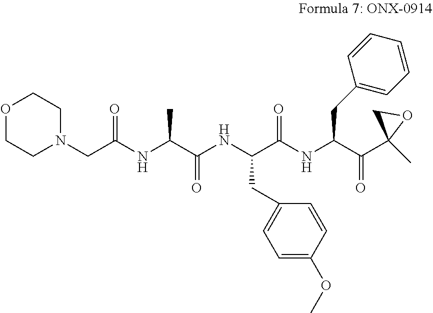 [SMILES: COc1ccc(C[C@H](NC(=O)[C@H](C)NC(=O)CN2CCOCC2)C(=O)N[C@@H](Cc2ccccc2)C(=O)[C@]2(C)CO2)cc1]

In a preferred example of the invention, the proteasome protein inhibitor is Epoxomicin, which has the structural formula shown in Formula 8:

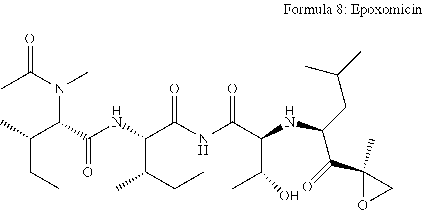 [SMILES: CC[C@H](C)[C@H](NC(=O)[C@H]([C@@H](C)CC)N(C)C(C)=O)C(=O)NC(=O)[C@@H](N[C@@H](CC(C)C)C(=O)[C@@]1(C)CO1)[C@@H](C)O]

In a preferred example of the invention, the proteasome protein inhibitor is VR23, which has the structural formula shown in Formula 9:

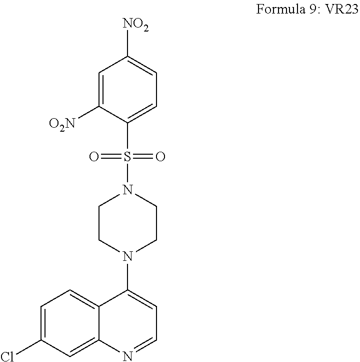 [SMILES: O=[N+]([O-])c1ccc(S(=O)(=O)N2CCN(c3ccnc4cc(Cl)ccc34)CC2)c([N+](=O)[O-])c1]

In a preferred example of the invention, the proteasome protein inhibitor is MLN-2238, which has the structural formula shown in Formula 10:

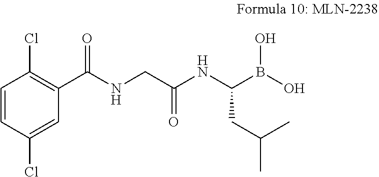 [SMILES: CC(C)C[C@H](NC(=O)CNC(=O)c1cc(Cl)ccc1Cl)B(O)O]

In a preferred example of the invention, the proteasome protein inhibitor is Celastrol, which has the structural formula shown in Formula 11:

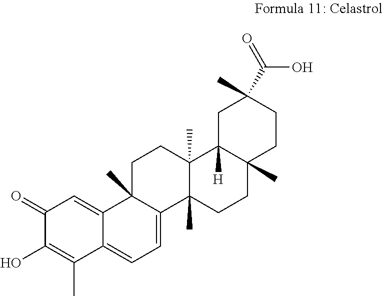

In a preferred example of the invention, the proteasome protein inhibitor is PI-1840, which has the structural formula shown in Formula 12:

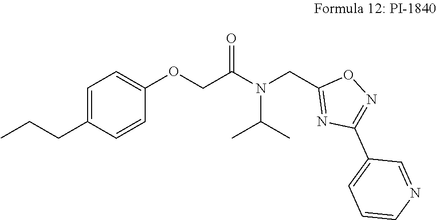 [SMILES: CCCc1ccc(OCC(=O)N(Cc2nc(-c3cccnc3)no2)C(C)C)cc1]

In some preferred examples of the invention, the proteasome inhibitor further comprises tools for inhibiting gene expression against any subunit of the proteasome, including, but not limited to, tools or materials for gene interference, gene silencing, and gene editing or gene knockout.

As an alternative embodiment, the tools for inhibiting gene expression are selected from one or more of DNA, RNA, PNA, DNA-RNA hybrids. They may be single-stranded or double-stranded.

Proteasome inhibitors may include small inhibitory nucleic acid molecules such as short interfering RNA (siRNA), double-stranded RNA (dsRNA), microRNA (miRNA), ribozymes, and small hairpin RNA (shRNA), all of which attenuate or eliminate gene expression of proteasome subunits.

These small inhibitory nucleic acid molecules may include first and second strands that hybridize to each other to form one or more double-stranded regions, each strand being about 18 to 28 nucleotides in length, about 18 to 23 nucleotides in length, or 18, 19, 20, 21, 22 nucleotides in length. Alternatively, a single strand may comprise regions capable of hybridizing to each other to form a double strand, such as in an shRNA molecule.

These small inhibitory nucleic acid molecules may include modified nucleotides while maintaining this ability to attenuate or eliminate proteasome expression. Modified nucleotides can be used to improve in vitro or in vivo properties, such as stability, activity, and/or bioavailability. These modified nucleotides may contain deoxynucleotides, 2′-methyl nucleotides, 2′-deoxy-2′-fluoronucleotides, 4′-trinucleotides, locked nucleic acid (LNA) nucleotides, and/or 2′-O-methoxyethyl nucleotides and others. Small inhibitory nucleic acid molecules, such as short interfering RNA (siRNA), may also contain 5′- and/or 3′-cap structures to prevent degradation by exonucleases.

In some examples, a double-stranded nucleic acid consisting of a small inhibitory nucleic acid molecule comprises blunt-ended, or overhanging nucleotides. Other nucleotides may include nucleotides that result in dislocations, bumps, loops, or wobble base pairs. Small inhibitory nucleic acid molecules can be formulated for administration, e.g., by liposome encapsulation, or incorporation into other carriers (e.g., biodegradable polymer hydrogels, or cyclodextrins).

In other preferred examples of the invention, the proteasome inhibitor further comprises one or more of antibodies, an antibody functional fragment, a peptide and a peptidomimetic. For example, an antibody, antibody functional fragment, peptide, or peptidomimetic that binds to any functional domain of any subunit of the proteasome. For example, any one or more of α₁₋₇ subunits and β₁₋₇; as a preferred embodiment, the antibody binds to a subunit of the proteasome core particle; as a more preferred embodiment, the antibody binds to β₁, β₂ or β₅ subunit of the proteasome.

Among them, the antibody may be a monoclonal antibody, a polyclonal antibody, a multivalent antibody, a multispecific antibody (for example: bispecific antibody), and/or antibody fragments linked to the proteasome. The antibody can be a chimeric antibody, a humanized antibody, a CDR-grafted antibody, or a human antibody. Antibody fragments can be, for example, Fab, Fab′, F(ab′)2, Fv, Fd, single chain Fv (scFv), FV with a disulfide bond (sdFv), or VL, VH domains. The antibody may be in a conjugated form, for example, conjugated to a label, a detectable label, or a cytotoxic agent. The antibody may be a isotype IgG (e.g., IgG1, IgG2, IgG3, IgG4), IgA, IgM, IgE or IgD.

In the invention, the alphavirus is selected from the following groups or mutants of the following groups: Eastern Equine Encephalitis virus, Venezuelan Equine Encephalitis virus, Everglades virus, Mucambo virus, Pixuna virus, Western Encephalitis virus, Sindbis virus, South African arbovirus No. 86, Girdwood S. A. virus, Ockelbo virus, Semliki Forest virus, Middleburg virus, Chikungunya virus, O'Nyong-Nyong virus, Ross River virus, Barmah Forest virus, Sagiyama virus, Bebaru virus, Mayaro virus, Una virus, Aura virus, Whataroa virus, Babanki virus, Kyzlagach virus, Highlands J virus, Fort Morgan virus, Ndumu virus, Buggy Creek virus, M1 virus, Getah virus, and any other viruses classified by the International Committee on Taxonomy of Viruses (ICTV) as an alphavirus.

As a preferred embodiment, the alphavirus is selected from the group consisting of M1 virus, Getah virus, or a combination thereof.

The alphavirus according to the present invention may in particular be referred to as viruses which are currently available, but do not exclude viruses in which natural variations or mutations (natural mutations, mandatory mutations, or selective mutations), genetic modifications, sequence additions or deletions or partial substitutions are possible. The alphavirus described herein include viruses that have been altered as described above. Preferably, the above-mentioned changes do not affect the function of the alphavirus according to the present invention. As an alternative embodiment, in the present invention, the alphavirus may be an intact virus or a nucleic acid molecule thereof; the nucleic acid molecule is derived from: single-stranded RNA or complementary DNA of alphavirus; or synthetic alphavirus RNA; alternatively, the alphavirus genome or portion thereof is capable of inducing cytolysis when administered to a cell or a subject.

The proteasome inhibitors are substances (e.g., compounds, or amino acid sequences, nucleotide sequences, etc.) or tools, etc. that act to knock down or affect proteasome gene expression or reduce the amount or activity of the proteasome. A person skilled in the art can modify, replace, change etc., the inhibitory compound or the gene tool thereof, but as long as the effect of inhibiting the proteasome is achieved, the proteasome inhibitor belongs to the proteasome inhibitor of the invention, and belongs to the homogeneous replacement of the substances, compounds or tools and the like.

As an alternative embodiment, the genome sequence of the alphavirus has at least 95%, at least 96%, at least 97%, at least 98%, at least 99%, at least 99.5%, at least 99.8%, or at least 99.9% identity to the sequence set forth in Genbank Accession No. EF011023.

As an alternative embodiment, the genome sequence of the alphavirus has at least 95%, at least 96%, at least 97%, at least 98%, at least 99%, at least 99.5%, at least 99.8%, or at least 99.9% identity to the genome sequence of the virus deposited under accession number CCTCC V201423.

In some examples, the alphavirus is the M1 virus deposited under accession number CCTCC V201423 (deposited at the China Center for Type Culture Collection on Jul. 17, 2014). As a virus likely to originate from the same strain, Genbank Accession No. EF011023 (Wen J S, Zhao W Z, Liu J W, et al. Genomic analysis of a Chinese isolate of Getah-like virus and its phylogenetic relationship with other Alphaviruses[J]. Virus genes, 2007, 35(3): 597-603.) recorded the sequence of a M1 strain. As a virus with up to 97.8% identity to M1 virus (Wen et al. Virus Genes. 2007; 35(3):597-603), Getah virus has high identity with M1 virus, and M1 virus has been classified as Getah-like virus in some literatures. In one embodiment, the alphavirus of the invention comprises an M1 or Getah virus having up to 97.8% or more sequence identity to the M1 strain.

Individual alphavirus strain may also be administered. In other embodiments, multiple strains and/or types of alphavirus may also be used.

The invention also provides a pharmaceutical composition comprising a proteasome inhibitor and an alphavirus.

The invention also provides a pharmaceutical kit for treating tumors, comprising the proteasome inhibitor, and the alphavirus.

As an embodiment, the pharmaceutical composition or pharmaceutical kit may further comprise a pharmaceutically acceptable carrier.

As one embodiment, the dosage form of the pharmaceutical composition or pharmaceutical kit includes, but is not limited to, a lyophilized powder injection, an injection, a tablet, a capsule, or a patch.

As a preferred embodiment, the pharmaceutical composition or pharmaceutical kit is in the form of an injection.

As one embodiment, the pharmaceutical composition or pharmaceutical kit is used to treat a tumor.

Pharmaceutical kits differ from compositions in that proteasome inhibitors differ from alphavirus dosage forms but are packaged separately (e.g.: pills, or capsules, or tablets or ampoules, containing proteasome inhibitors; additional pills, or capsules, or tablets or ampoules, containing alphavirus). In some examples, the alphavirus, proteasome inhibitor, and combination of alphavirus and proteasome inhibitor may also contain one or more adjuvants. The adjuvant refers to a component which can assist the therapeutic effect of the medicament in the pharmaceutical composition. The pharmaceutical kit may also contain separately packaged proteasome inhibitors, as well as separately packaged alphavirus. The administration of the proteasome inhibitor, as well as the alphavirus, in the pharmaceutical kit can be simultaneous or in any anteroposterior order, or cross-administration, such as administration of the proteasome inhibitor before the alphavirus, or administration of the proteasome inhibitor after the alphavirus, or both. In various examples, the patient may be a mammal. In some examples, the mammal may be a human.

In addition, the pharmaceutical kit further comprises instructions for using the kit according to the combined therapy of the present invention.

The invention also provides use of the combination of the proteasome inhibitor and the alphavirus in the preparation of a medicament for treating tumors.

The present invention also provides a method of treating a tumor by separately, sequentially, simultaneously, jointly or sequentially cross, administering to a patient having a tumor in need of treatment an effective amount of the proteasome inhibitor and an effective amount of the alphavirus.

As a preferred embodiment, the proteasome inhibitors include, but are not limited to, compounds such as Bortezomib, Carfilzomib and Oprozomib that inhibit proteasome protein activity. Or, tools for inhibiting gene expression of the proteasome, including, but not limited to, tools or materials for gene interference, gene silencing, and gene editing or gene knockout.

As a preferred embodiment, in the composition, pharmaceutical kit or method of treatment, the proteasome inhibitor is selected from the following compounds or derivatives thereof having proteasome inhibitory effect, or pharmaceutically acceptable salts, solvates, tautomers, isomers thereof: Bortezomib, Carfilzomib, MG-132, ONX-0914, ONX-0912, CEP-18770, MLN-9708, Epoxomicin, VR23, MLN-2238, Celastrol and PI-1840. As a more preferred embodiment, the proteasome inhibitor may preferably be Bortezomib, Carfilzomib or a combination thereof.

In general, techniques and protocols for administering medicaments are known in the art.

The proteasome inhibitor and/or alphavirus of the present invention are administered intraperitoneally, intravenously, intra-arterially, intramuscularly, intradermally, intratumorally, subcutaneously or intranasally.

As a preferred embodiment, the proteasome inhibitor and/or alphavirus are administered intravenously.

In the present invention, alphavirus administration by intratumoral or intravenous injection significantly inhibits tumor growth. The only oncolytic virus medicament currently available in Europe and America, T-Vec, is administered intratumorally to treat melanoma. Compared with intravenous injection, the administration mode requires special training of doctors and nurses, the acceptance of patients is not high, and the administration mode is not suitable for deep organ tumors and micrometastases. The alphavirus in the invention can be treated by intravenous administration, which is more convenient and feasible in clinical application.

As a preferred embodiment, in the use, composition, pharmaceutical kit or method, the following amounts of alphavirus are contained or administered: at least 10¹ viral particles or PFU; Preferably 10¹-10³⁰ viral particles or PFU; More preferably, 10¹, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹ or 10²² viral particles or PFU.

As a preferred embodiment, in the use, composition, pharmaceutical kit or method, the proteasome inhibitor is contained or administered in an amount of 0.01 to 2000 mg; preferably 0.01 to 1000 mg; preferably 0.01 to 500 mg; preferably 0.01 to 200 mg; preferably 0.1 to 200 mg; preferably 0.1 to 100 mg.

As a preferred embodiment, the ratio of proteasome inhibitor (e.g., Bortezomib, Carfilzomib, Oprozomib, etc.) to alphavirus is optionally: 0.01 to 200 mg:10³ to 10⁹ PFU; preferably 0.1 to 200 mg:10⁴ to 10⁹ PFU; more preferably 0.1 to 100 mg:10⁵ to 10⁹ PFU.

Preferably the dosages used are: proteasome inhibitors (e.g., Bortezomib, Carfilzomib, or Oprozomib, etc.) are used in the range of 0.01 mg/kg to 200 mg/kg, while alphaviruses are used with a titer at MOI from 10³ to 10⁹ (PFU/kg); preferably proteasome inhibitors (e.g., Bortezomib, Carfilzomib or Oprozomib, etc.) are used in the range of 0.1 mg/kg to 200 mg/kg, while alphaviruses are used with a titer at MOI from 10⁴ to 10⁹ (PFU/kg); more preferably, proteasome inhibitors (e.g., Bortezomib, Carfilzomib, or Oprozomib, etc.) are used in the range of 0.1 mg/kg to 100 mg/kg, while alphaviruses are used with a titer at MOI from 10⁵ to 10⁹ (PFU/kg).

In the present invention, the tumor is any tumor; in one embodiment, the tumor is a solid tumor or a hematological tumor. In particular, the solid tumors are adrenocortical carcinoma, pararenocortical carcinoma, anal carcinoma, appendiceal carcinoma, astrocytoma, atypical teratoma, rhabdomyoma, basal cell carcinoma, cholangiocarcinoma, bladder cancer, bone cancer, brain tumor, bronchial tumor, Burkett's lymphoma, carcinoid tumor, heart tumor, bile duct epithelial carcinoma, chordoma, colorectal cancer, craniopharyngioma, ductal carcinoma in situ, embryonal tumor, endometrial carcinoma, ependymoma, esophageal carcinoma, olfactory neuroblastoma, intracranial embryonic cell tumor, extragonadal germ cell tumor, eye cancer, carcinoma of the fallopian tube, gallbladder carcinoma, head and neck cancer, hypopharyngeal carcinoma, Kaposi's sarcoma, renal carcinoma, Langerhans cell histiocytosis, laryngeal carcinoma, lip cancer, oral cancer, Meckel cell carcinoma, malignant mesothelioma, multiple endocrine neoplasia syndrome, mycosis fungoides, nasal sinus carcinoma, neuroblastoma, non-small cell lung cancer, ovarian cancer, pancreatic neuroendocrine tumor, islet cell tumor, papillomatosis, paraganglioma, carcinoma of nasal sinus and nasal cavity, parathyroid carcinoma, carcinoma of penis, carcinoma of pharynx and larynx, pituitary tumor, pleuropulmonary blastoma, primary peritoneal carcinoma, retinoblastoma, salivary gland tumor, sarcoma, Sezary syndrome, skin cancer, small cell lung cancer, small intestinal carcinoma, soft tissue sarcoma, squamous cell carcinoma, testicular cancer, thymoma and thymic carcinoma, thyroid cancer, urethral cancer, uterine cancer, endometrium and uterine sarcoma, vaginal carcinoma, vascular tumor, vulvar carcinoma, solitary myeloma, liver cancer, colorectal cancer, bladder cancer, breast cancer, cervical cancer, prostate cancer, glioma, melanoma, pancreatic cancer, nasopharyngeal cancer, lung cancer or gastric cancer.

As a preferred embodiment, the hematological tumor is acute lymphoblastic leukemia, acute myelocytic leukemia, chronic lymphocytic leukemia, chronic myelogenous leukemia, lymphoma or multiple myeloma;

As a preferred embodiment, the solid tumors are liver cancer, colorectal cancer, bladder cancer, breast cancer, cervical cancer, prostate cancer, glioma, melanoma, pancreatic cancer, nasopharyngeal cancer, lung cancer or gastric cancer insensitive to alphavirus.

In a preferred embodiment, the tumor is a tumor insensitive to alphavirus.

In a preferred embodiment, the tumors are liver cancer, colorectal cancer, bladder cancer, breast cancer, cervical cancer, prostate cancer, glioma, melanoma, pancreatic cancer, nasopharyngeal cancer, lung cancer or gastric cancer insensitive to alphavirus.

In a preferred embodiment, the tumor is a tumor insensitive to M1 oncolytic virus.

The invention finds that the proteasome inhibitor can increase the anti-tumor effect of the alphavirus so as to improve the therapeutic effectiveness of the alphavirus as an anti-tumor medicament. The cytological experiment proves that the combined use of the M1 virus and the proteasome inhibitor can obviously cause morphological lesions of tumor cells, thus significantly enhance the inhibitory effect on tumor cells.

We combined Bortezomib and M1 virus to treat human hepatocellular carcinoma Hep3B strain and Huh 7 strain. It have been surprisingly found that the combined use of Bortezomib and M1 virus significantly increases the morphological changes of tumor cells and significantly decreases the viability of tumor cells. For example, in one embodiment of the present invention, when M1 virus (MOI=0.1) is used to treat hepatoma cell Huh 7 alone, the tumor cell viability is 78.7%, when 5 nM Bortezomib is used to treat tumor cells, the tumor cell viability is still as high as 99.7%, and when 5 nM Bortezomib is used in combination with M1 virus of the same MOI (MOI=0.1), the tumor cell viability decreased to 35.7%. Compared with the anti-tumor effect of using M1 virus alone, the oncolytic effect of Bortezomib combined with M1 was significantly improved. It can be seen that the greatly enhanced oncolytic effect of Bortezomib combined with M1 is due to the synergistic mechanism between Bortezomib and M1 virus, not simply through the anti-tumor mechanism of Bortezomib.

The inventors previously used chrysophanol and derivatives thereof as anti-cancer synergist of M1 virus, and found through the experiment that the viability of tumor cells decreased to 39.6% after the combination of 50 μM chrysophanol and M1 virus, while the invention found that the viability of tumor cells decreased significantly to 35.7% after the combination of 5 nM Bortezomib and M1 virus. Compared with chrysophanol and derivatives thereof, the M1 anti-tumor synergist of the invention significantly improves the killing rate of tumor, at the same time, the pharmaceutical effective dose of Bortezomib is only 1/10000 of that of chrysophanol, and the effect is fast, and the time used is ⅔ of that of chrysophanol (72 h by chrysophanol treatment and 48 h by Bortezomib treatment), which has significant advantages.

### BRIEF DESCRIPTION OF THE DRAWINGS

FIG. 1 Bortezomib and M1 virus significantly increased morphological lesions of human hepatocellular carcinoma strains.

FIG. 2 Treatment of Bortezomib combined with M1 virus significantly reduced the viability of human hepatocellular carcinoma strains.

FIG. 3 Carfilzomib or treatment combined with M1 virus significantly inhibited the growth of human hepatocellular carcinoma strain xenograft tumor; wherein FIG. 3A is a flow chart of medicament treatment time; FIG. 3B shows that the treatment of Carfilzomib combined with M1 virus significantly inhibited the growth of human hepatocellular carcinoma strain Hep3B xenograft tumor; FIG. 3C shows that the treatment of Carfilzomib combined with M1 virus significantly inhibited the growth of human hepatocellular carcinoma strain Huh 7 xenograft tumor.

FIG. 4 Treatment of proteasome inhibitors combined with M1 virus significantly reduced the viability of human hepatocellular carcinoma strains; FIG. 4A shows that the treatment of CEP-18770 or combined with M1 virus significantly inhibits the viability of human hepatocellular carcinoma strains; FIG. 4B shows that MLN-9708 or treatment combined with M1 virus significantly inhibits the viability of human hepatocellular carcinoma strains; FIG. 4C shows that ONX-0912 or treatment combined with M1 virus significantly inhibited human hepatocellular carcinoma viability.

### DETAILED DESCRIPTION

The following embodiments further illustrate the present invention, but the embodiments of the present invention are not limited to the following examples. Any equivalent changes or modifications made in accordance with the principles or concepts of the present invention should be regarded as the scope of protection of the present invention.

Without being specifically indicated, the materials and experimental methods employed in the present invention are conventional materials and methods.

The term “selected from” in the specification is used in connection with a selected object and is to be understood as, for example: “X is selected from: A, B, C, . . . , E” or “X is selected from one or more of A, B, C, . . . and E”, and the like, are understood to mean that X includes one of A, B, C, E, or any combination of both, or any combination of more. At this time, it is not excluded that X also includes some other types of substances.

In the present invention, the singular forms “a, an”, and “the” include plural form unless the context clearly dictates otherwise.

In the present invention, “treat” refers to alleviation of symptoms, temporary or permanent elimination of the cause of symptoms, or prevention or alleviation of the symptoms of a given disease or disorder.

In the present invention, “pharmaceutically acceptable carrier” refers to molecular entities and compositions that do not produce an allergic or similar adverse reaction when administered to a human. Including any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Except insofar as any conventional media or agent is incompatible with the active ingredient, the use thereof in the therapeutic compositions is contemplated.

In the present invention, a “pharmaceutically acceptable salt” is prepared by reacting the free acid or base form of a compound with a suitable base or acid in water or in an organic solvent or in a mixture of the two. Including acid addition salts or base addition salts. Examples of acid addition salts include inorganic acid addition salts such as hydrochloride, hydrobromide, hydroiodide, sulfate, nitrate, phosphate, and organic acid addition salts such as acetate, trifluoroacetate, maleate, fumarate, citrate, oxalate, succinate, tartrate, malate, mandelate, methanesulfonate, and p-toluene sulfonate. Examples of base addition salts include inorganic salts such as sodium salt, potassium salt, calcium salt and ammonium salt, and organic base salts.

The term “effective amount” includes an amount of an alphavirus or proteasome inhibitor used in the present invention sufficient to provide the desired therapeutic effect. The exact amount required will vary from subject to subject, depending on factors such as: the species being treated, the age and general condition of the subject, the severity of the condition being treated, the particular agent being administered and the mode of administration, and the like. However, for a given situation, the dosage of the pharmaceutical compositions of the present invention may be adjusted by an ordinary skill in the art according to the severity of the symptoms, the frequency of recurrence, and the physiological response of the treatment regimen.

In addition to the above-mentioned proteasome inhibitors, the proteasome inhibitors of the present invention may be selected from proteasome inhibitors already known in the art, or substances found to have proteasome inhibition effect through subsequent studies. Examples of proteasome inhibitors include, but are not limited to, the following groups: Bortezomib, Carfilzomib, MG-132, ONX-0914, ONX-0912 (Oprozomib), CEP-18770 (Delanzomib), MLN-9708 (Ixazomib), Epoxomicin, VR23, MLN-2238, Celastrol, PI-1840, [(1R)-1-({[(2, 3-difluorobenzoyl) amino] acetyl} amino)-3-methylbutyl] boronic acid, [(1R)-1-({[(2, 3-difluorobenzoyl) amino] acetyl} amino)-3-methylbutyl] boronic acid, [(1R)-1-({[(5-chloro-2-fluorobenzoyl) amino] acetyl} amino)-3-methylbutyl] boronic acid, [(1R)-1-({[(3, 5-difluorobenzoyl) amino] acetyl} amino)-3-methylbutyl] boronic acid, [(1R)-1-({[(2, 5-difluorobenzoyl) amino] acetyl} amino)-3-methylbutyl] boronic acid, [(1R)-1-({[(2-bromobenzoyl) amino] acetyl} amino)-3-methylbutyl] boronic acid, [(1R)-1-({[(2-fluorobenzoyl) amino] acetyl} amino)-3-methylbutyl] boronic acid, [(1R)-1-({[(2-chloro-5-fluorobenzoyl) amino] acetyl} amino)-3-methylbutyl] boronic acid, [(1R)-1-({[(4-fluorobenzoyl) amino] acetyl} amino)-3-methylbutyl] boronic acid, [(1R)-1-({[(3, 4-difluorobenzoyl) amino] acetyl} amino)-3-methylbutyl] boronic acid, [(1R)-1-({[(3-chlorobenzoyl) amino] acetyl} amino)-3-methylbutyl] boronic acid, [(1R)-1-({[(2, 5-dichlorobenzoyl) amino] acetyl} amino)-3-methylbutyl] boronic acid, [(1R)-1-({[(3, 4-dichlorobenzoyl) amino] acetyl} amino)-3-methylbutyl] boronic acid, [(1R)-1-({[(3-fluorobenzoyl) amino] acetyl} amino)-3-methylbutyl] boronic acid, [(1R)-1-({[(2-chloro-4-fluorobenzoyl) amino] acetyl} amino)-3-methylbutyl] boronic acid, [(1R)-1-({[(2, 3-dichlorobenzoyl) amino] acetyl} amino)-3-methylbutyl] boronic acid, [(1R)-1-({[(2-chlorobenzoyl) amino] acetyl} amino)-3-methylbutyl] boric acid, [(1R)-1-({[(2, 4-difluorobenzoyl) amino] acetyl} amino)-3-methylbutyl] boric acid, [(1R)-1-({[(4-chloro-2-fluorobenzoyl) amino] acetyl} amino)-3-methylbutyl] boric acid, [(1R)-1-({[(4-chlorobenzoyl) amino] acetyl} amino)-3-methylbutyl] boric acid, [(1R)-1-({[(2, 4-dichlorobenzoyl) amino] acetyl} amino)-3-methylbutyl] boronic acid, [(1R)-1-({[(3, 5-dichlorobenzoyl) amino] acetyl} amino)-3-methylbutyl] boronic acid, and mannitol esters, salts thereof or boronic acid anhydrides: which can also be found in patents WO07017440, EP11195107, U.S. 60/683,385, U.S. Ser. No. 09/300,779A, U.S. 60/815,218, WO04026407, and U.S. 60/495,764, all of which are incorporated herein by reference.

Depending on the type of tumor and the stage of disease progression, the effects of the tumor treatment methods of the present invention include, but are not limited to, inhibiting tumor growth, delaying tumor growth, tumor regression, tumor contraction, and increasing regeneration time of tumor when the treatment stops, slowing down the progression of the disease and preventing metastasis.

### Example 1 Bortezomib and M1 Viruses Significantly Increased Morphological Lesions in Human Hepatoma Cell Strains

Materials:

Human hepatocellular carcinoma Hep3B (purchased from ATCC) and Huh 7 (purchased from ATCC), M1 virus (Accession No. CCTCC V201423), high glucose DMEM medium (purchased from Corning), inverted phase contrast microscope.

Method:

a) Cell culture: human hepatocellular carcinoma Hep3B and Huh 7 were grown in DMEM complete medium containing 10% FBS, 100 U/ml penicillin and 0.1 mg/ml streptomycin; all cell strains were cultured and passaged in a closed incubator at a constant temperature of 37° C. (95% relative humidity) with 5% CO₂, and the growth was observed under inverted microscope. The cells were passaged about every 2 to 3 days, and the cells in the logarithmic phase were used in the formal experiment.

b) Cell treatment and morphological observation: cells in the logarithmic growth phase and the DMEM complete culture medium (containing 10% fetal bovine serum and 1% double antibody) were selected to prepare cell suspension, and the cells were inoculated in a 24-well culture plate at a density of 2.5×10⁴/well. The cells were treated with Bortezomib (5 nM) alone, infected with M1 virus (Hep3B: 0.001 moi, Huh 7: 0.1 moi) and treated with M1 virus combined with Bortezomib, with neither M1 virus nor Bortezomib as control, the morphological changes of cells were observed under inverted phase contrast microscope after 48 h.

Results:

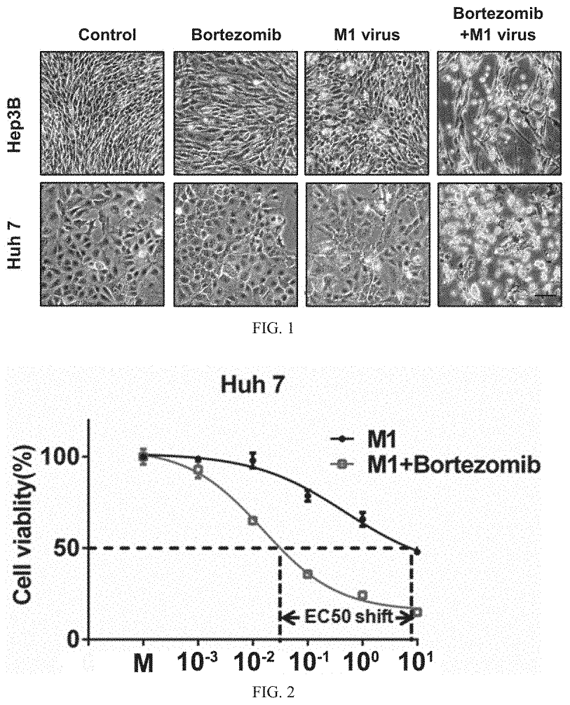

As shown in FIG. 1, the cell morphology was observed under a phase contrast microscope, the Hep3B cells and Huh 7 cells in the control group grew in a monolayer with close arrangement and consistent phenotype, and there was no significant change in cell morphology after 48 h of treatment with Bortezomib (5 nM) or M1 virus (Hep3B: 0.001 moi, Huh 7: 0.1 moi), respectively. However, after 48 h of treatment by Bortezomib combined with M1 virus, compared with the control group and each single treatment group, the number of cells in the combined treatment group decreased significantly, and the morphology of the cells changed significantly, the cell body contracted into a ball, and the refractive index increased significantly, showing a death lesion.

### Example 2 Treatment of Bortezomib Combined with M1 Virus Significantly Reduced the Viability of Human Hepatoma Cell Strains

Materials:

Human hepatocellular carcinoma Huh 7 (purchased from ATCC), M1 virus (Accession No. CCTCC V201423), high glucose DMEM medium (purchased from Corning), automatic enzyme-linked detection microplate reader.

Method:

a) Inoculating cells, and drug administering treatment: cells in the logarithmic growth phase and the DMEM complete culture medium (containing 10% fetal bovine serum and 1% double antibody) were selected to prepare cell suspension, and were inoculated in a 96-well culture plate at a density of 4×10³/well. 12 h later, the cells were completely adhered to the wall. The cells were divided into control group without medicament nor virus treatment, Bortezomib treatment group, M1 infection group and Bortezomib/M1 combined group. The doses used were as follows: M1 virus (MOI=0.001, 0.01, 0.1, 1, 10) infected cells, Bortezomib was 5 nM.

b) MTT reacted with intracellular succinate dehydrogenase: after culturing for 48 h, 20 μl (5 mg/ml) of MTT was added to each well and incubated for 4 h, at this time, the granular bluish violet formazan crystals formed in living cells could be observed under microscope.

c) Dissolving formazan granules: the supernatant was carefully removed, DMSO 100 μl/well was added to dissolve the crystal, shaked in a microoscillator for 5 min, and then detect the optical density (OD value) of each well with wavelength of 570 nm on the enzyme-linked detector. The experiment was repeated for 3 times in each group. Cell viability=OD value of medicament treatment group/OD value of control group×100%.

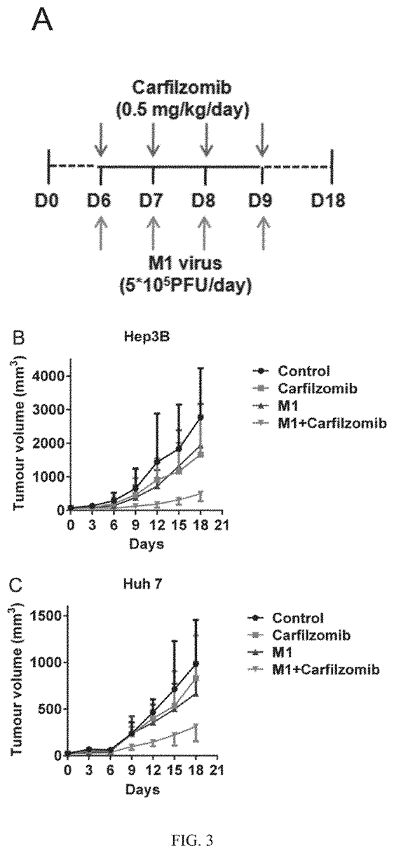

d) Origin 8 was used for nonlinear curve fitting, and two dose-response curves were drawn with drug dose as abscissa and relative cell viability as ordinate, namely, the dose-response curve of M1 virus alone and the dose-response curve of Bortezomib combined with M1 virus. The EC50 shift of the two curves was calculated, that is, the EC50 shift in FIG. 2, the larger the difference value was, the more significant the medicament synergized.

Results:

As shown in FIG. 2, treatment with Bortezomib (5 nM) alone had a small inhibitory effect on the viability of tumor cells Huh 7, and the relative cell viability of tumor cells reached 99.7%, the relative cell viability of the group treated with M1 virus (MOI=0.1) was still as high as 78.7%. However, when the same 5 nM Bortezomib was combined with M1 virus (MOI=0.1) (Eeyarestatin I+M1), the relative cell viability of tumor cells decreased significantly to 35.7%. Compared with single treatment, different doses of M1 virus (MOI=0.001, 0.01, 0.1, 1, 10) combined respectively with Bortezomib (5 nM) significantly decreased the viability of tumor cell Huh 7.

### Example 3 Carfilzomib Combined with M1 Virus Significantly Inhibited the Growth of Human Hepatoma Cell Strain Xenograft Tumor

Materials:

M1 virus (accession number CCTCC V201423), human hepatoma cell strain Hep3B (purchased from ATCC), human hepatoma cell strain Huh 7 (purchased from ATCC), 4-week-old female BALB/c nude mice.

Method:

This experiment adopts a random, single-blind design. 5×10⁶ Hep 3B or Huh 7 cells were injected subcutaneously into the dorsal side of 4-week-old BALB/c nude mice. When the tumor size reached 50 mm³, the mice were grouped including untreated control group, Carfilzomib group (intraperitoneal injection 0.5 mg/kg/d), M1 infected group (tail vein injection of M1 virus 5×10⁵ PFU per time) and Carfilzomib/M1 combined group (the same dose of Carfilzomib and M1 virus was given in the same way), which were injected consecutively for 4 times in four days (see FIG. 3A). The length, width and body weight of the tumor were measured every two days, and the volume of the tumor was according to the formula (length×width 2)/2.

Results:

In two kinds of tumor cells (human hepatoma cell strain Hep3B and human hepatoma cell strain Huh 7) xenograft tumor animals, pathological anatomic measurements of tumor volume showed that, compared with the control group, Carfilzomib group and M1 infected group could only cause a slight reduction in tumor volume, while Carfilzomib/M1 combined group could cause a significant reduction in tumor volume (FIGS. 3B and 3C). At the end of the experiment, in the human hepatoma cell strain Hep3B model, the tumor volume of the control group was 2772.5 mm², the tumor volume of Carfilzomib group and M1 infected group were 1668.5 mm² and 1940 mm², while the tumor volume of Carfilzomib/M1 combined group was 499 mm². In the human hepatoma cell strain Huh 7 model, the tumor volume of the control group was 983.5 mm², the tumor volume of the Carfilzomib group and the M1 infected group were 830.5 mm² and 667.0 mm², while the tumor volume of the Carfilzomib/M1 combined group was 313.7 mm². One way ANOVA statistics showed that the difference was statistically significant (FIGS. 3B and 3C).

### Example 4 Treatment of Various Proteasome Inhibitors Combined with M1 Virus Significantly Reduced the Viability of Human Hepatoma Cell Strains

Materials:

Human hepatocellular carcinoma Huh 7 (purchased from ATCC), M1 virus (Accession No. CCTCC V201423), high glucose DMEM medium (purchased from Corning), automatic enzyme-linked detection microplate reader.

Method:

a) Inoculating cells, and drug administering treatment: cells in the logarithmic growth phase and the DMEM complete culture medium (containing 10% fetal bovine serum and 1% double antibody) were selected to prepare cell suspension, and were inoculated in a 96-well culture plate at a density of 4×10³/well. 12 h later, the cells were completely adhered to the wall. The cells were divided into control group without medicament nor virus treatment, proteasome inhibitor group (including CEP-18770, MLN-9708, ONX-0912), M1 infected group and proteasome inhibitor/M1 combined group. The doses used were as follows: the doses used were as follows: M1 virus (MOI=0.1) infected cells; Proteasome inhibitor doses are as follows: CEP-18770 (5 nM), MLN-9708 (5 nM), ONX-0912 (50 nM).

b) MTT reacted with intracellular succinate dehydrogenase: after culturing for 72 h, 20 μl (5 mg/ml) of MTT was added to each well and incubated for 4 h, at this time, the granular bluish violet formazan crystals formed in living cells could be observed under microscope.

c) Dissolving formazan granules: the supernatant was carefully removed, DMSO 100 μl/well was added to dissolve the crystal, vibrate in a microoscillator for 5 min, and then detect the optical density (OD value) of each well with wavelength of 570 nm on the enzyme-linked detector. Cell viability=OD value of medicament treatment group/OD value of control group x 100%.

Results:

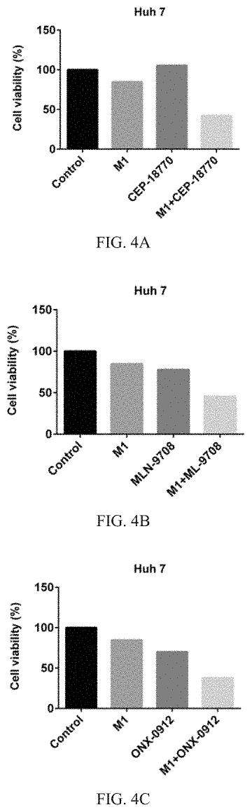

As shown in FIG. 4A, treatment with CEP-18770 alone had a small effect on the viability of tumor cells Huh 7, and the relative cell viability of tumor cells reached 105.4%, the relative cell viability of the group treated with M1 virus (MOI=0.1) was still as high as 84.6%. However, when the same CEP-18770 was combined with M1 virus (MOI=0.1), the relative cell viability of tumor cells decreased significantly to 42.2%; as shown in FIG. 4B, treatment with MLN-9708 alone had a small effect on the viability of tumor cells Huh 7, and the relative cell viability of tumor cells reached 77.7%, the relative cell viability of the group treated with M1 virus (MOI=0.1) was still as high as 84.6%. However, when the same CEP-18770 was combined with M1 virus (MOI=0.1), the relative cell viability of tumor cells decreased significantly to 45.3%; as shown in FIG. 4C, treatment with ONX-0912 alone had a small effect on the viability of tumor cells Huh 7, and the relative cell viability of tumor cells reached 70.0%, the relative cell viability of the group treated with M1 virus (MOI=0.1) was still as high as 84.6%. However, when the same CEP-18770 was combined with M1 virus (MOI=0.1), the relative cell viability of tumor cells decreased significantly to 37.8%.

The described embodiments of the present invention are merely illustrative examples, and the embodiments of the present invention are not limited to the above, and any other changes, modifications, substitutions, combinations, and simplifications that may be made without departing from the spirit and principles of the present invention shall be equivalent replacement and shall be included in the protection scope of the present invention.

1-10. (canceled)

11. A method for treating a tumor in a subject in need thereof, comprising:
administering to the subject in need thereof
an effective amount of a proteasome inhibitor, and
an effective amount of an alphavirus.

12. The method of claim 11, wherein the alphavirus is selected from the group consisting of Eastern Equine Encephalitis virus, Venezuelan Equine Encephalitis virus, Everglades virus, Mucambo virus, Pixuna virus, Western Encephalitis virus, Sindbis virus, South African arbovirus No. 86, Girdwood S. A. virus, Ockelbo virus, Semliki Forest virus, Middleburg virus, Chikungunya virus, O'Nyong-Nyong virus, Ross River virus, Barmah Forest virus, Sagiyama virus, Bebaru virus, Mayaro virus, Una virus, Aura virus, Whataroa virus, Babanki virus, Kyzlagach virus, Highlands J virus, Fort Morgan virus, Ndumu virus, Buggy Creek virus, M1 virus and Getah virus.

13. The method of claim 11, wherein the alphavirus is selected from at least one of M1 virus and Getah virus.

14. The method of claim 11, wherein the alphavirus is M1 virus.

15. The method of claim 11, wherein
the genome sequence of the alphavirus has at least 95% identity to the sequence indicated by Genbank Accession No. EF011023; and/or
the genome sequence of the alphavirus has at least 95% identity to the genome sequence of the virus deposited under accession No. CCTCC V201423.

16. The method of claim 11, wherein the proteasome inhibitor is a substance that inhibits proteasome activity, or inhibits the activity or expression of any one subunit of the proteasome, or blocks assembly of proteasome subunits, or degrades the proteasome.

17. The method of claim 11, wherein the proteasome inhibitor is selected from a group consisting of:
Bortezomib, Carfilzomib, MG-132, ONX-0914, ONX-0912, CEP-18770, MLN-9708, Epoxomicin, VR23, MLN-2238, Celastrol and P1-18400;
or derivatives thereof having proteasome inhibitory effect, or pharmaceutically acceptable salts, solvates, tautomers, isomers thereof.

18. The method of claim 11, wherein the proteasome inhibitor is selected from a group consisting of:
Bortezomib, Carfilzomib, MG-132, ONX-0914, ONX-0912, CEP-18770 and MLN-9708;
or derivatives thereof having proteasome inhibitory effect, or pharmaceutically acceptable salts, solvates, tautomers, isomers thereof.

19. The method of claim 11, wherein the proteasome inhibitor is selected from gene interference, gene editing, gene silencing or gene knockout materials.

20. The method of claim 11, wherein the proteasome inhibitor is selected from one or more of DNA, RNA, PNA and DNA-RNA hybrids.

21. The method of claim 11, wherein the proteasome inhibitor is selected from one or more of siRNA, dsRNA, miRNA, shRNA and ribozyme.

22. The method of claim 11, wherein the proteasome inhibitor is a tumor targeting proteasome inhibitor.

23. The method of claim 11, wherein the tumor is a solid tumor or a hematological tumor.

24. The method of claim 11, wherein the tumor is selected from a group consisting of:
adrenocortical carcinoma, pararenocortical carcinoma, anal carcinoma, appendiceal carcinoma, astrocytoma, atypical teratoma, rhabdomyoma, basal cell carcinoma, cholangiocarcinoma, bladder cancer, bone cancer, brain tumor, bronchial tumor, Burkett's lymphoma, carcinoid tumor, heart tumor, bile duct epithelial carcinoma, chordoma, colorectal cancer, craniopharyngioma, ductal carcinoma in situ, embryonal tumor, endometrial carcinoma, ependymoma, esophageal carcinoma, olfactory neuroblastoma, intracranial embryonic cell tumor, extragonadal germ cell tumor, eye cancer, carcinoma of the fallopian tube, gallbladder carcinoma, head and neck cancer, hypopharyngeal carcinoma, Kaposi's sarcoma, renal carcinoma, Langerhans cell histiocytosis, laryngeal carcinoma, lip cancer, oral cancer, Meckel cell carcinoma, malignant mesothelioma, multiple endocrine neoplasia syndrome, mycosis fungoides, nasal sinus carcinoma, neuroblastoma, non-small cell lung cancer, ovarian cancer, pancreatic neuroendocrine tumor, islet cell tumor, papillomatosis, paraganglioma, carcinoma of nasal sinus and nasal cavity, parathyroid carcinoma, carcinoma of penis, carcinoma of pharynx and larynx, pituitary tumor, pleuropulmonary blastoma, primary peritoneal carcinoma, retinoblastoma, salivary gland tumor, sarcoma, Sézary syndrome, skin cancer, small cell lung cancer, small intestinal carcinoma, soft tissue sarcoma, squamous cell carcinoma, testicular cancer, thymoma and thymic carcinoma, thyroid cancer, urethral cancer, uterine cancer, endometrium and uterine sarcoma, vaginal carcinoma, vascular tumor, vulvar carcinoma, solitary myeloma, liver cancer, colorectal cancer, bladder cancer, breast cancer, cervical cancer, prostate cancer, glioma, melanoma, pancreatic cancer, nasopharyngeal cancer, lung cancer and gastric cancer.

25. The method of claim 11, wherein the tumor is selected from a group consisting of:
liver cancer, colorectal cancer, bladder cancer, breast cancer, cervical cancer, prostate cancer, glioma, melanoma, pancreatic cancer, nasopharyngeal cancer, lung cancer and gastric cancer.

26. The method of claim 11, wherein the tumor is selected from a group consisting of:
acute lymphoblastic leukemia, acute myelocytic leukemia, chronic lymphocytic leukemia, chronic myelogenous leukemia, lymphoma or multiple myeloma.

27. The method of claim 11, wherein the tumor is insensitive to alphavirus.

28. The method of claim 11, wherein the ratio of said proteasome inhibitor and said alphavirus is 0.01 to 200 mg: 10³ to 10⁹ PFU.

29. The method of claim 11, wherein
0.01 mg/kg to 200 mg/kg of said proteasome inhibitor is administered; and
a titer at MOI from 10³ to 10⁹ PFU/kg of said alphavirus is administered.

30. The method of claim 11, wherein
said proteasome inhibitor is administered intraperitoneally, intravenously, intra-arterially, intramuscularly, intradermally, intratumorally, subcutaneously or intranasally; and/or
said alphavirus is administered intraperitoneally, intravenously, intra-arterially, intramuscularly, intradermally, intratumorally, subcutaneously or intranasally.
